(19) United States
(12) Patent Application Publication
(10) Pub. No.: US 2009/0235923 A1
(43) Pub. Date: Sep. 24, 2009

(54) SPRAY DEVICE AND NOZZLE CLOSURE

(75) Inventor: Billy Nilson, Mjolby (SE)

Correspondence Address: VENABLE LLP, P.O. BOX 34385, WASHINGTON, DC, 20043-9998, US

(73) Assignee: MEDUX AB, Mjolby (SE)

(21) Appl. No.: 12/299,427

(22) PCT Filed: Apr. 27, 2007
(86) PCT No.: PCT/SE2007/050285

§ 371 Date: Nov. 3, 2008

(30) Foreign Application Priority Data
May 2, 2006 (SE) 0600957-5

(51) Int. Cl. B05B 11/04; B05B 1/30; A61M 15/08; B21D 53/00

(52) U.S. Cl. 128/200.14; 239/533.13; 222/632; 222/494; 29/890.1

(57) ABSTRACT

A spray device including a casing and a spray bottle. A closing element is arranged in a nozzle. The closing element substantially fills the nozzle in order to minimize residual amounts of a drug in the nozzle. The overall purpose is to protect the spray device from contamination and evaporation. A method for manufacturing with as few parts as possible a spray device that by usually manual action causes the spray bottle to effect a pumping movement which triggers a spray dose through the nozzle. The device is intended to increase dose accuracy and facilitate the actual pumping.

### TECHNICAL FIELD

The present invention relates to a spray device with a nozzle closure, intended primarily for spraying a drug from a liquid container. The invention further relates to a method for spraying by means of the spray device and a method for manufacturing the device.

### STATE OF THE ART

A known spray device in common everyday use comprises a liquid container which is gripped with a thumb under the bottom of the liquid container and a finger on each side of a nozzle connected to the liquid container. By pressing the thumb against the bottom of the liquid container and applying an opposite grip to a pump activator at the nozzle end of the liquid container and thereby exerting a pressure in the axial direction of the liquid container, such a spray device may be caused to delivery a quantity of liquid, e.g. to deliver a specific dose of a drug to a nasal cavity. The spray device may then be difficult to handle, since the axial movement may cause unintentional movement of the nozzle within the nasal cavity, thereby possibly disturbing the dosage or rendering it ineffective. Moreover, the hand grip is troublesome because it involves at least three fingers, one at the bottom and two on the pump activator. For users with small or handicapped hands, e.g. rheumatics, it may be difficult or simply impossible to effect such a form of pumping movement. A person with thick fingers may find it difficult to place the nozzle far enough into the nasal cavity because of fingers possibly being in the way. Spray devices of this type thus have disadvantages.

Devices for protection against contamination in spray devices are described in, for example, U.S. Pat. No. 6,722,585 and U.S. Pat. No. 5,988,499. The embodiment according to U.S. Pat. No. 5,988,449 constitutes in its simplest form a relatively complicated device made of relatively large amounts of a number of different materials, with consequent environmental disadvantages. U.S. Pat. No. 6,722,585 takes the form of a structure comprising an elastomer (a plurality of different materials), which is inappropriate from the destruction point of view and hence from the environmental point of view. US 2004/0245291 is a publication referring to a dispenser with friction-loaded activation arms and nozzle in integral form.

The dispenser lacks protection against contamination in the nozzle. Said inventions have large nozzles, which means that relatively large residual amounts of drug may remain in the nozzle after each use. A disadvantage of long nozzles and the consequently relatively large volumes in them is that the spray device may need “priming” before it is used, i.e. by a user effecting spraying into surrounding air to make sure that the drug really does fully emerge from the nozzle. Priming is also necessary, at least after a time, in the case of nozzles with no closure. Such a procedure contaminates the air in the surroundings, which is very disadvantageous, particularly where care staff effect this priming repeatedly, perhaps many times per day, and are thus regularly exposed to unnecessary inhalation of one or more active drugs.

Spray devices have existed for decades and are used in very large numbers throughout the world, so seemingly small changes in a positive direction as regards both function and simplified production technology and reduced input of materials may be of very great significance.

An object of the present invention is to offer solutions to the disadvantages described above of spray devices according to the state of the art.

### BRIEF DESCRIPTION OF THE INVENTION

One aspect of the invention proposes a device with characteristics according to the attached claim 1.

A further aspect of the invention proposes a method with characteristics according to the attached independent method claim.

A yet further aspect of the invention is indicated by the independent claim indicating a method for manufacturing the spray device.

Further embodiments of the invention are presented in the dependent claims.

Modern drugs are often active pharmaceutical substances and it is therefore important that no priming should be needed. The fact that no priming is needed with the present invention is a very important advantage of the invention. Priming involves a possibly empty nozzle and adjoining components being initially filled with liquid, which often entails activating the spray bottle a couple of times. This nearly always results in a spray cloud being atomised out into the surrounding air space, which may cause harm to staff exposed to it.

A simple and reliable design proposed according to the present invention represents an improvement step which may be crucial to the existence of the present technology on the market and perhaps a final step for improvement relative to a previously totally dominant design which has considerable disadvantages for users because of involving an awkward grip on the bottom of the container, two fingers on the container's “shoulders” and the need for repeated priming with consequent incorrect dosage and contamination of surrounding air.

The invention comprises a casing with an activation device for a spray bottle and with a short nozzle, with integral manufacture of the casing, the activation device and the nozzle. The short nozzle is provided with a space-filling insert to minimise the residual amount of fluid (liquid). The insert is also provided with a shutoff in the nozzle to prevent contamination. When fitted with a spray bottle, the spray device constitutes a precision spray which is easy to activate. Being made integrally with a short nozzle and activation arms which facilitate activation is particularly advantageous from the functional and manufacturing points of view. Improvements reducing the number of parts which have to be manufactured and fitted may be of very great significance where the number of units on the market exceeds several hundred million per annum.

Known spray devices are provided with long nozzles, whereas the device according to the present invention has a short nozzle, thereby reducing the residual amount of a drug in liquid form which remains in the nozzle after use. The insert proposed according to the invention further reduces the residual amount of liquid. Short nozzle in this context may mean a maximum length in the axial direction of not more than 15 mm.

The expression “spray bottle” hereinafter means a liquid container intended to accommodate a liquid drug, and an associated pump, when the liquid constituting the drug is at substantially atmospheric pressure. Spray bottle may also mean an aerosol bottle containing a liquid drug which is subjected to a considerably higher than atmospheric pressure by a propellant gas enclosed in the liquid container. Pumping hereinafter means a regular pumping movement to activate the pump so that it actively pumps the liquid to an outlet pipe for the spray bottle in a known manner. If the spray bottle is of aerosol type, pumping means triggering a valve to allow the liquid acted upon by the pressure in the bottle to move through said outlet pipe for the spray bottle.

An object of the present invention is to provide a spray device which eliminates or reduces the disadvantages of previously known devices and can easily be used by anyone, particularly by persons who have difficulties in effecting activation of the spray bottle.

Another object of the invention is to provide improved manual pumping/triggering of the spray bottle whereby forces are applied mainly perpendicular to the main direction of flow of the liquid and the axial direction of the nozzle, and the spray bottle is simpler to operate through being easier to handle, since the user can use the whole hand.

In a preferred embodiment, the casing has areas on its outside wall which are formed as clamping surfaces to which the directional forces have to be applied.

The invention is characterised by its simplicity and small number of parts. The present invention thus comprises a device for delivering a specified amount of liquid in spray form and comprises a casing, with accommodation for a spray bottle, and a nozzle through which a flow of the liquid passes in order to form an aerosol, and an activating device. The activating device, hereinafter called the activator, comprises the aforesaid clamping surfaces and at least one link arm extending between the lower portion of the clamping surfaces and provided with a “knee” which is adapted to moving axially towards the nozzle when in contact with the bottom of the spray bottle as a result of the application to the clamping surfaces of two forces (P) in opposite directions substantially perpendicular to the direction of the nozzle.

In a preferred embodiment, the activator extends from the casing. As previously mentioned, the spray bottle introduced into the spray device comprises a container for a liquid and a pump/valve which transfers a specific volume of liquid under pressure from the spray bottle to the nozzle which is integrated with the upper portion of the spray device, i.e. integrated with the casing, whereby said volume of liquid converts to a cloud of aerosols leaving the nozzle.

In a preferred embodiment, the outlet from the casing is formed as a pipe, constituting the actual nozzle, in which there is a device for breaking up the liquid to form a finely divided spray, which pipe is further adapted to accommodating a cylindrical insert in an internally cylindrical space in which a piston can run freely. The piston is provided at its one end with a shutoff valvepiece, preferably conical in shape, which fits in a seat at the orifice of the nozzle. The cylindrical insert fits tightly against the tubular inside wall of the nozzle.

At least one groove, preferably two or more, run(s) along the inside of the pipe in the axial direction of the nozzle. When the insert is placed within the nozzle and thereby fits to the tubular inside wall of the nozzle, said grooves become a corresponding number of ducts, which communicate with the outlet pipe of the spray bottle and with the orifice to the nozzle. The liquid which passes from the spray bottle via the ducts converts to drop form as it passes through the orifice.

The insert has a movable piston which is acted upon by the liquid pressure on its upper portion so that the orifice to the nozzle is freed, as a result of the liquid from the ducts exerting a downward pressure on the piston so that the latter moves away from the orifice, thereby moving the shutoff cone away from the orifice, which thus opens. In the absence of liquid pressure, the piston is urged by springs towards the orifice, which is then closed by the shutoff cone.

In another embodiment, the insert comprises a movable piston whose conical surface closes the outlet of the spray bottle and which is suspended resiliently by springs which act upon a surface on the inside of the very topmost portion of the nozzle.

### DESCRIPTION OF THE DRAWINGS

FIGS. 1a-1c depict the spray device from all directions and FIG. 1d depicts it in cross-section with a spray bottle fitted.

FIG. 2 depicts the casing and the nozzle with the activating device before the fitting of a spray bottle.

FIG. 3 depicts a variant of the casing.

FIG. 4 depicts the nozzle with the insert and piston at their uppermost position, closing the orifice.

FIG. 5 depicts the nozzle with the insert and piston at their lowermost position, freeing the orifice for spraying.

FIG. 6 depicts a variant of the nozzle with the insert and piston at their uppermost position, closing the orifice.

FIG. 7 depicts the nozzle variant according to FIG. 6 with the insert and piston at their lowermost position, freeing the orifice for spraying.

FIG. 8 depicts a cross-section of the nozzle at the section A-A in FIG. 4.

FIG. 9 depicts the same as FIG. 8 but at the section B-B in FIG. 5.

FIG. 10 depicts the nozzle with a valvepiece which closes the pump's orifice with a conical seal.

FIG. 11 depicts the same nozzle as in FIG. 10 but with the valvepiece lifted by the liquid pressure from the spray bottle and therefore in an open position.

FIG. 12 depicts a cross-section according to section A-A in FIG. 10 showing the path of the liquid at one point. For the sake of clarity, the path of the liquid via the other three alternatives is not depicted.

FIG. 13 depicts a nozzle with a fixed insert, which constitutes a simplified variant of a spray nozzle in cases where no barrier to contamination is deemed desirable.

FIG. 14 depicts a variant insert valvepiece in the nozzle with the valvepiece in closed position.

FIG. 15 depicts a same variant as FIG. 14 with the valvepiece in open position.

FIG. 16 depicts part of the outward appearance of the valvepiece.

### DESCRIPTION OF EMBODIMENTS

A number of embodiments of the invention are described below with reference to the attached drawings.

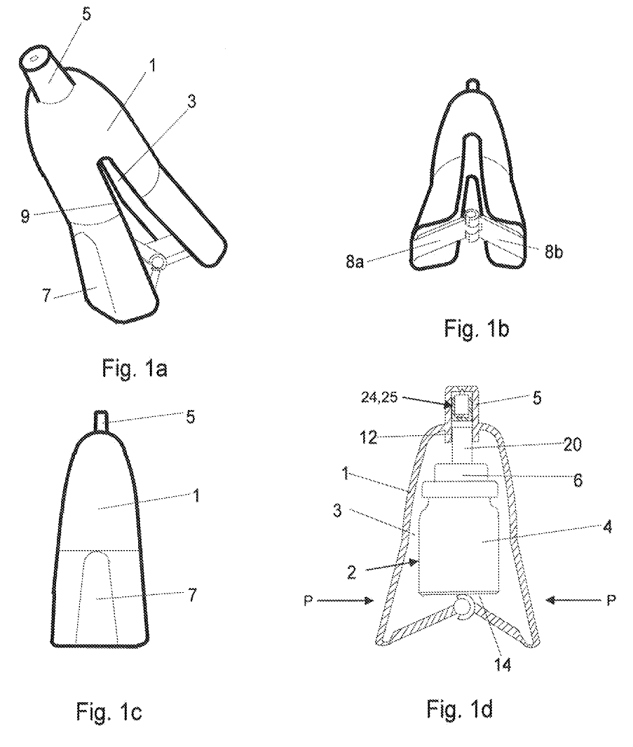

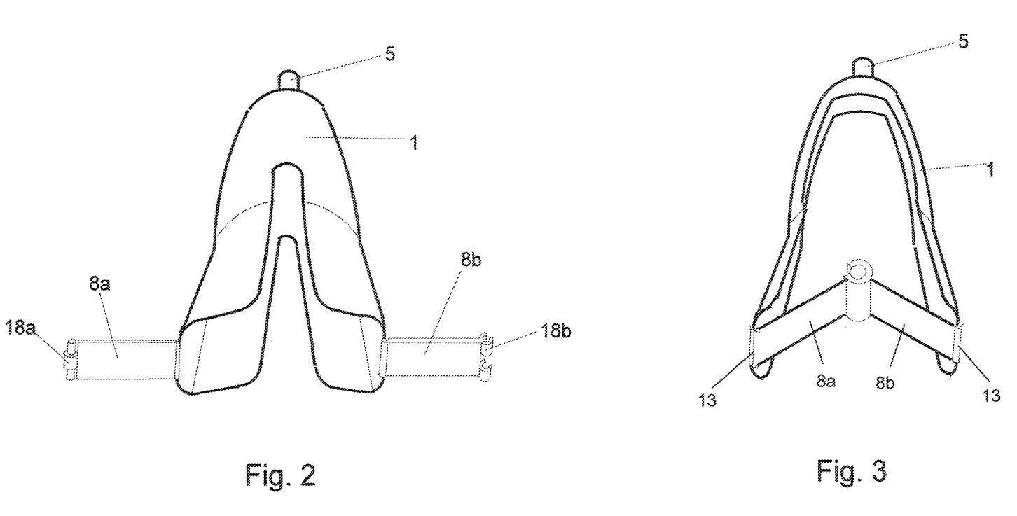

The spray device in FIGS. 1a-1d and 2 comprises a casing 1 made of plastic, e.g. polypropene, and is provided with a hollow space 3 for accommodating and containing a spray bottle 2, and an outlet 5 in the form of a nozzle via which a dose preferably of liquid is sprayed. FIGS. 1a to 1c depict the casing 1 before the spray bottle 2, in this case in the form of a liquid container 4 with a pump 6, is fitted, and FIG. 1d depicts the spray device ready for use.

In the preferred embodiment in FIG. 2, the casing is usually a tubular circular section which is slightly conical, with the cone's centreline coinciding with the nozzle's centreline. The casing thus has a broad bottom portion and a narrower top portion, at the outlet. The casing comprises integrally, from the top downwards, the nozzle 5, the casing 1 and the link arms 8a and 8b. At least one clamping surface 7 (see FIG. 1a) is formed in the wall at the lower portion of the casing. In a preferred embodiment, however, the casing 1 is provided with a slit 9 and with two opposite clamping surfaces 7 which extend upwards for a distance on the casing and are designed for manual pressure in a safe and grip-friendly manner. The spray device also comprises the link arms 8a and 8b. The clamping surfaces 7 fit together with the link arms 8a and 8b to form what is called the activator intended to activate a dose from the spray bottle 2 when the latter is fitted in the casing 1.

The nozzle 5 starts from the casing and has an orifice 11 in the form of an aperture in the foremost portion of the nozzle. The nozzle is preferably integral with the casing 1. On the inside of the casing there is a tubular seat 12 to which in this example the pump 6 with an associated liquid container 4 is fitted. The activator is preferably provided with link arms 8a and 8b which are equal in length and each integral with the casing 1 and also comprises the pair of clamping surfaces 7. When the spray bottle 2 is fitted, the link arms 8a and 8b are first snapped together with a snap lock 18a, 18b, after which they are turned in at a material thinning 13 on the respective side. The link arms thus form an angle with one another and thereby further lock the spray bottle 2 securely at its bottom 14 so that it cannot fall out of the casing 1. The spray device is then ready for action.

FIG. 1d depicts the spray device as a whole in the form of a cross-section with the spray bottle 2 comprising a liquid container 4, preferably a bottle, and a manually activated pump 6 which is fastened to the bottle and delivers a specified dose when it is activated. The spray bottle 2 is fitted in the casing 1 in the hollow space 3. The pump 6 comprises a pump body, not depicted, and a tubular portion 20 (also called the outlet pipe) which is introduced into the lower/inner portion of the nozzle 5 in a recess in the form of the seat 12. The nozzle 5, through which the intended dose of a liquid is sprayed when the tubular portion 20 is pressed into the pump 6, extends outwards from the casing 1 in an axial direction. In practice, this means that the liquid container 4 is pushed upwards by the activator, thereby pressing the tubular portion 20 into the pump device, when a force P presses the clamping surfaces 7 together towards one another.

If the spray bottle 2 takes the form of an aerosol container, the intended dose of a liquid is sprayed when the tubular portion 20 is pressed against a trigger for a valve (not depicted) in the aerosol container in order to release liquid which is under pressure in the container. In practice this means that the spray bottle 2 is pushed upwards by the activator whereby the tubular portion 20 presses in the valve's trigger, when a force P presses the clamping surfaces 7 together towards one another, whereby a mist jet of the liquid in the spray bottle is created at the orifice 11. The variant with a spray bottle in the form of an aerosol container in combination with a valve is not depicted in the drawings but may be exemplified by the drawings already presented, where the aerosol container is comparable with ref. 4 and the aerosol container's valve with trigger is comparable with ref. 6.

FIG. 2 depicts the casing 1 with the link arms 8a and 8b turned outwards. FIG. 1b depicts the casing 1 fitted with the snap locks 18a and 18b locked together and forming a “knee”, but without having the spray bottle 2 inserted in the casing.

To effect spraying of a specified dose, the force P is applied to the opposite clamping surfaces 7, with the result that the “knee” formed by the link arms bends further upwards and thereby lifts the liquid container 4, this movement being considerably facilitated by the slit 9 in the walls of the casing. This lifting movement activates the pump 6, which thereupon pushes the intended amount of liquid out through the nozzle, in which an atomising device breaks up the liquid to form an aerosol. In the case of an aerosol bottle, it is not the pump but the trigger of the valve which is activated.

Alternative embodiments are feasible, e.g. an ellipsoidal or spherical shape of casing or a clamping surface 7 on one side opposite to a fixed portion. Another example is making the casing in the form of a framelike strip which is of approximately the same width as the link arms 8a and 8b and may be said to constitute a continuation of them round the spray bottle 2 and with the nozzle uppermost, i.e. a more open embodiment of the casing according to this aspect of the invention. This embodiment displays the contents of the spray bottle 2 and providing the latter with a graduation scale makes it possible to count the number of doses.

The casing 1 may alternatively be provided with a wall which has a frame structure or mesh structure. Such a embodiment of the casing affords the advantage that the spray bottle housed by the casing is readily identifiable by a user, who will therefore easily be able to check which type of drug or the like is contained in the casing. This advantage may of course also be achieved by using transparent plastic for the casing 1.

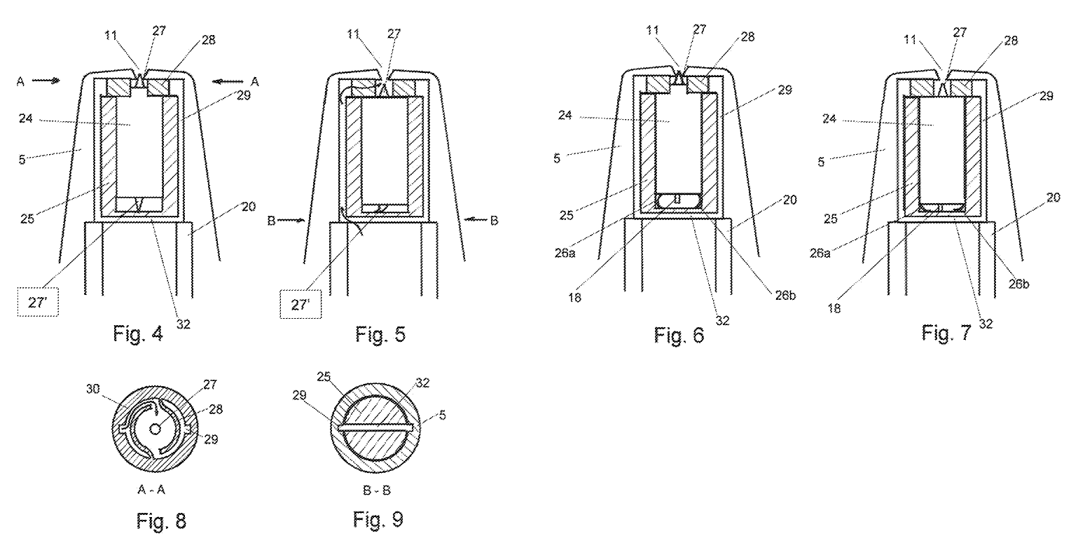

A simultaneously sealing and space-filling element of the nozzle 5 is constituted by an insert comprising a piston 24 and a sleeve 25 in FIG. 4. As previously mentioned, the nozzle is provided with inside walls of a cylindrical form. Assembly involves the sleeve 25 being pushed into the nozzle from inside and fitting tightly to the inside walls of the nozzle. The sleeve 25 is not pushed right up to a surface formed internally in the forward part of the nozzle but is caused to leave a space between the sleeve 25 and the nozzle's forward inside wall by at least one spacer 28. The piston 24 is movable and runs in a cylindrical space in the sleeve 25 whereby the piston 24 in a fitted state closes the nozzle's orifice 11 by means of a valvepiece 27. The piston 24 has a similar valvepiece 27′ at its lower end, which means that the piston 24 can be fitted with either end towards the orifice 11. The valvepiece 27′, which points towards the spray bottle 2 and bears against the bottom of the sleeve 25, has the property of constituting a resilient component and serves at the same time as a distance stop for the piston 24, the downward movement of which is therefore limited by this stop provided by the valvepiece 27′. FIG. 5 depicts the piston 24 at its lower position in which it is acted upon by the pump pressure on its upper end and therefore frees the orifice 11 for spraying.

At least one groove 29, preferably two or more grooves 29, run(s) along the cylindrical walls which constitute the inside of the nozzle, in the axial direction of the nozzle. When the insert, in the form of the sleeve 25 and the piston 24, is fitted in the nozzle 5 and is thus adjacent to the nozzle's tubular inside wall, said grooves 29 become a corresponding number of ducts. These ducts 29 (the same reference notation as the grooves) communicate with the outlet pipe 20 of the spray bottle and with the orifice 11 of the nozzle 5. Communication between the ducts 29 and the orifice 11 is effected by the spacer or spacers 28 being so configured as to provide communication between the ducts 29 and the orifice 11 via at least one formed outlet duct 30. Liquid which passes from the spray bottle 2 via the ducts 29 converts to drop form as it passes through the orifice 11. The outlet duct/ducts 30 may be given a desired shape in order to create a desired propagation pattern for the aerosol formed outside the orifice 11 by a mist jet of the liquid, which is created in the orifice.

A variant with a simultaneously resilient and sealing device is depicted in FIGS. 14 to 16. In this embodiment, the valvepiece 27 is provided with a bent-downwards flange with a bent-downwards annular lip 34 which bears on the upper end of the sleeve 27. Liquid pressure from the spray bottle 2 pushes the valvepiece 27 downwards as depicted in FIG. 15, whereby the nozzle's orifice 11 is freed and can communicate with the ducts 29. For the sake of clarity, the spacers 28 appear in FIGS. 14 and 15 in merely chain-dotted form. The annular lip 34 on the flange constitutes both a sealing and a resilient element.

FIGS. 6 and 7 depict a variant where the lower valvepiece 27′ has its lower portion replaced by a spacer pin 18. Resilient elements 26a and 26b in the lower portion of the piston 24 cause the latter to spring back when the liquid pressure on the top of the piston decreases. The resilient elements take the form of any desired type of springs, e.g. leaf springs, coil springs or any kind of elastic elements. The springs are preferably made of plastic material.

To achieve an effective propagation pattern for the mist jet, the path of the liquid is arranged according to the cross-sectional depiction A-A in FIG. 8. Like the other drawings, FIG. 8 depicts in an axial direction the ducts 29 which, as previously mentioned, may take the form of one or more ducts to allow liquid from the spray bottle 2 to run past the sleeve 25 on its way towards the orifice 11 of the nozzle 5. FIG. 9 depicts a embodiment of how liquid passes the underside of the sleeve 25 (forming part of the insert) via a transverse duct 32 on its way out to the ducts 29. The transverse duct 32 thus constitutes the duct which provides communication for liquid from the spray bottle 2 to the ducts 29, so the positions and number of the transverse ducts 32 have to be adapted to the positions and number of the ducts 29.

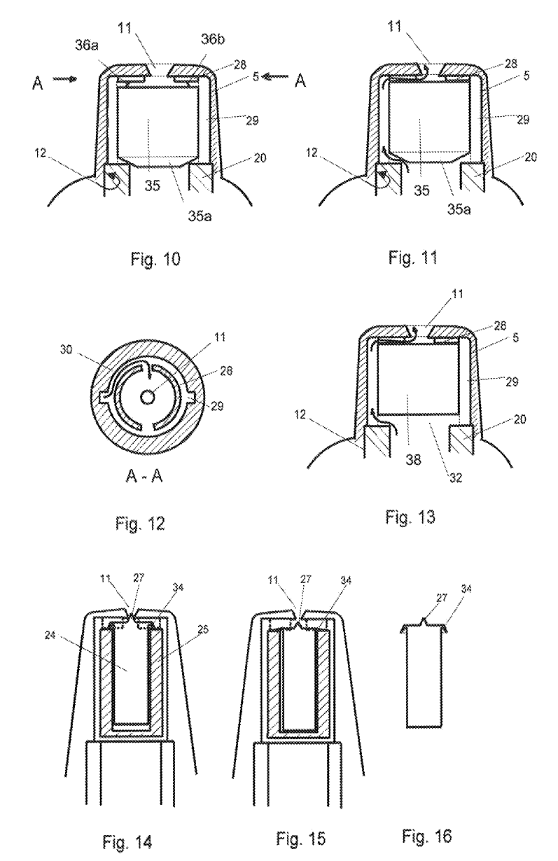

FIG. 10 and FIG. 11 depict a variant of an insert in the nozzle 5 where in this case the insert lacks a feature corresponding to the sleeve 25 and has instead a piston 35 which runs in the cylinder formed by the cylindrical inside walls of the nozzle 5. According to this embodiment, springs 36a and 36b are disposed between the upper portion of the piston and the forward inside wall of the nozzle 5, i.e. at the orifice 11. In this example the piston 35 has a cone 35a which seals against the spray bottle's outlet pipe, and when no spraying is activated the springs 36a and 36b urge the piston 25 towards the outlet pipe 20.

FIG. 12 depict a variant of the cross-section A-A in FIG. 4 where the spacers 28 are configured to cause rotation of the liquid before it reaches the orifice 11. The spacers 28 hold the ducts 32 open to allow liquid to pass.

FIG. 13 depicts a embodiment with a fixed insert in the form of a non-movable piston 38. In this case one end surface of the piston 38 is in a fixed position relative to the nozzle's forward inside wall, i.e. at the orifice. Here again, spacers 28 are disposed between the piston 38 and the nozzle's inside wall so that the piston 38 cannot close the orifice. In this case the purpose of the piston 38 is to fill the inside space of the nozzle within the inside walls of the nozzle 5, in order thereby to provide the least possible space for liquid in the outer portion of the nozzle 5 and simultaneously provide a bearing surface for the liquid during spraying in order to effect an advantageous whirling. This embodiment represents a simplified variant of a spray nozzle in cases where no barrier to contamination is deemed necessary/desirable but where the residual amount of liquid in the nozzle is nevertheless minimised by the fact that the insert in the form of the piston 38 substantially fills the space between the inside walls of the nozzle 5.

### Definition

Cylindrical space means any cylinder according to mathematical definition.

1. A device for spraying a specific amount of a liquid, the device comprising:
a casing comprising a hollow space; and
a nozzle for accommodating a spray bottle in the hollow space of the casing,
wherein the casing comprises an activating device comprising two opposite side surfaces comprising clamping surfaces on a lower portion of the casing, and at least one link arm integrally articulated with the side surfaces,
wherein said link arms are intended to be turned in towards a bottom of the spray bottle when the spray bottle has been inserted in the casing and is accommodated in the hollow space, whereby forces applied in opposite directions to the side surfaces convert to an axial movement of the activating device which pushes the spray bottle towards the nozzle,
wherein the casing, the nozzle and the activating device are formed integrally, and
wherein to minimize a residual amount of liquid in the nozzle, the comprises an insert which substantially fills a space formed by inside walls of the nozzle.

2. The device according to claim 1, wherein the walls of the nozzle are axially directed, wherein the inside walls of the nozzle comprise at least one groove whereby at least one duct is formed between said inside walls and the insert, and wherein said at least one duct connects to a transverse duct which communicates with an outlet pipe of the spray bottle.

3. The device according to claim 2, further comprising:
at least one spacer arranged between an upper end of the insert and a forward inside wall of the nozzle, wherein said forward inside wall comprises an orifice, whereby the at least one spacer is configured such that at least one outlet duct is formed in one of the at least one spacer, whereby said at least one duct communicates with the orifice when the spray device is activated.

4. The device according to claim 3, wherein the insert comprises a cylindrical sleeve comprising a cylindrical space inside the sleeve in which a piston can run in an axial direction, and wherein the sleeve is fixed and fits to axially directed inside walls of the nozzle.

5. The device according to claim 4, wherein the piston has on a first base surface a valvepiece which seals against an orifice of the nozzle.

6. The device according to claim 5, wherein the piston comprises a resilient element which is situated on an upper end of the piston and which bears against an upper end of the sleeve.

7. The device according to claim 5, wherein the piston comprises resilient elements which from a bottom in the sleeve exert a spring force against a second base surface of the piston whereby the piston is urged towards and seals against the orifice.

8. The device according to claim 7, wherein the resilient element comprises any element out of the following group: a valvepiece comprising an elastic material of a same type as the sealing valvepiece on the first base surface of the piston, a leaf spring, a coil spring, or an element made of elastic material.

9. The device according to claim 8, wherein the resilient element is integrated with the piston.

10. The device according to claim 3, wherein the insert comprises a piston which fits to the axially directed inside walls of the nozzle, and wherein the piston can run in an axial direction along said inside walls.

11. The device according to claim 10, wherein the piston comprises a cone which seals against the outlet pipe of the spray bottle as a result of resilient elements disposed between the forward inside walls of the nozzle and exerting a spring force which urges the piston towards the outlet pipe.

12. The device according to claim 3, wherein the insert comprises a piston which substantially fills a space formed between the inside walls of the nozzle, and wherein the piston is in a fixed position such that the ducts communicate continuously with the outlet pipe in the spray bottle and with the orifice.

13. The device according to claim 1, wherein said at least one link arm of the activating device is adapted to locking the entrance to the hollow space of the casing.

14. The device according to claim 13, wherein slits on opposite sides of the casing on each side of the side surfaces weaken the casing at said slits and facilitate the pumping movement by reducing the forces required.

15. The device according to claim 1, wherein said at least one link arm of the activating device comprises weakenings at bending points.

16. The device according to claim 15, wherein the at least one bendable link arm of the activating device is fastened to a respective side surface of the casing by snappable hinge brackets to form a movable double arm with a knee which abuts against a bottom of the spray bottle.

17. The device according to claim 1, wherein the casing comprises a frame or mesh device surrounding the container.

18. A method for spraying a dose of a liquid from a spray bottle, the method comprising:
providing a casing comprising a nozzle integrated with the casing and a hollow space to accommodate the spray bottle,
introducing the spray bottle into said hollow space,
applying forces in opposite directions to an outside of the casing at lower portions of the casing in the form of side surfaces configured as clamping surfaces,
converting the oppositely directed forces with an activating device to a force exerted against a bottom of the spray bottle in substantially an axial direction of the spray bottle,
arranging an insert in such a way as to substantially fill a space formed by inside walls of the nozzle, and
relieving said axial forces by pumping a liquid which flows from the spray bottle to the nozzle, whereby pressure from the liquid pushes a piston belonging to the insert against a spring force acting on the piston so that an orifice in the forward inside wall of the nozzle opens and a mist jet of the liquid is sprayed out through said orifice.

19. The method according to claim 18, further comprising:
the liquid being arranged to flow from an outlet of the spray bottle via ducts comprising axially directed grooves in inside walls of the nozzle and adjacent walls of the insert, and
the liquid being arranged to flow from said ducts along an upper end of the piston, whereby the piston is pushed down by the pressure of the liquid on its upper end, whereby a valvepiece which is disposed on the upper end of the piston and seals the orifice is freed and opens the orifice.

20. The method according to claim 18, further comprising:
the liquid being arranged to flow from an outlet of the spray bottle, after which the liquid under pressure acts upon the underside of a piston which by spring force abuts against and seals the outlet and which itself constitutes said insert, and wherein an cylindrical wall of the piston thus abuts against the axially directed inside walls of the nozzle,
the piston acted upon by the pressure of the liquid being pushed upwards and opening the outlet so that the liquid can flow via ducts comprising axially directed grooves in the inside walls of the nozzle and adjacent walls of the piston and reach the orifice.

21. A method for manufacturing a spray device, the method comprising:
injection molding the spray device, a casing, a nozzle and said at least one link arm, being made of plastic and injection-moulded integrally and
press fitting said insert in situ between the axially directed inside walls of the nozzle.

22. The device according to claim 3, wherein the device comprises a plurality of spacers, and wherein the spacers are-configured such that at least one outlet duct is formed in or between the spacers.
